(19) United States
(12) Patent Application Publication
(10) Pub. No.: US 2018/0369516 A1
(43) Pub. Date: Dec. 27, 2018

(54) Device For Controlled Dispensation Of Liquid Medicament And Method Of Vaporization Control

(71) Applicant: Resolve Digital Health Inc., Toronto (CA)

(72) Inventor: Robert ADELSON, Vaughn (CA)

(73) Assignee: Resolve Digital Health Inc., Toronto (CA)

(21) Appl. No.: 16/018,851

(22) Filed: Jun. 26, 2018

Related U.S. Application Data
(60) Provisional application No. 62/525,522, filed on Jun. 27, 2017.

(51) Int. Cl. A61M 15/00

(52) U.S. Cl. CPC A61M 15/0025; A61M 15/009; A61M 15/06; A61M 15/0086; A61M 2202/03; A61M 15/0071

(57) ABSTRACT

The present disclosure relates to a drop dispenser device comprising: a liquid reservoir for holding a liquid medicament to be vaporized; a dispensing opening through which the liquid medicament is adapted to pass during a dispensing operation of the dispenser; and a dispensing mechanism configured to generate predetermined droplets of the liquid medicament, and expel the generated droplets through the dispensing opening in a selected liquid amount and at a selected frequency, upon activation of the dispensing mechanism.

### FIELD OF THE INVENTION

The present disclosure relates to a medicament dispensing device for use in therapeutic vaporizers. In particular, the present disclosure relates to a device capable of dispensing precise, replicable and consistent metered drops of liquid medicament for subsequent vaporization by dose vaporizer.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art

Therapeutic vaporizers provide an alternative drug-delivery method that permits patient to inhale vaporized medication rather than swallow a pill, or drink or inject medication. A variety of vaporizers and inhalation devices for vaporizing herbal oils and medicated liquids for pulmonary delivery have been developed and some of them are commercially available. These devices generally utilize drop dispensing systems that dispense liquid medication in droplet form to a heating assembly for vaporization. However, the drop volume or size produced by the existing dispensing mechanism is often inconsistent and non-replicable leading to inaccurate dosing of medications. As the dosage provided by a therapeutic vaporizer dependent upon the size of drops produced, drop dispensers that produce drops having sizes different than the desired or expected drop size may in turn yield medicament dosages that differ from the calculated and expected dosage. Further, the existing vaporizers or liquid dispensing systems do not provide for a method to control the volume of liquid that is dispensed for vaporization nor the volume of vapor produced and, as a result, cannot provide consistent and measurable doses of vaporized medication for pulmonary administration.

Further, the existing vaporizers or liquid dispensing systems fail entirely to provide provisions for monitoring and recording critical information including size and number of drops dispensed for vaporization, and for analyzing the gathered data in a way that would be useful for either therapy or in a clinical setting. Another disadvantage of the prior art devices is that they provide no provision for communication with external networks or other functional units for recording critical data relating to usage of the medicament dispensing system and dosage of medicinal product for subsequent clinical analysis.

Accordingly, it would be advantageous to provide a device that dispenses precise, replicable and consistent metered drops of liquid medication, and ensures that the dispensed drops can be vaporized in a manner that provides patients with a predictable dose. Another advantage would be to provide a liquid dispensing device with features to monitor and record size and number of drops dispensed for vaporization and number of doses remaining in the device thereby enhancing patient clinical care. Yet another advantage would be to provide a liquid dispensing device that is communicable in a web enabled environment to implement an effective medication management system.

The present invention satisfies the existing needs, as well as others, and overcomes the deficiencies found in the prior art.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all groups used in the appended claims.

### SUMMARY

In an aspect, the present disclosure relates to a drop dispenser device comprising: a liquid reservoir for holding a liquid medicament to be vaporized; a dispensing opening through which the liquid medicament is adapted to pass during a dispensing operation of the dispenser; and a dispensing mechanism configured to generate predetermined droplets of the liquid medicament, and expel the generated droplets through the dispensing opening in a selected liquid amount and at a selected frequency, upon activation of the dispensing mechanism.

In another aspect, the drop dispenser dispenses accurate metered droplets of the liquid medicament on a consistent and replicable basis to a heating assembly for subsequent evaporation. In another aspect, the liquid medicament can be a herbal oil such as cannabis oil/extract.

In another aspect, the device can enable monitoring and recording of size and number of droplets dispensed for vaporization and number of doses remaining in the dispenser.

In yet another aspect, the device can enable maintenance of records of droplet consumption on a central storage device such as on a cloud/server, wherein the records are accessible, from the central storage device, to one or more stakeholders based on permissions granted by user of said device.

In another aspect, the records can be automatically processed to enable determination of impact of droplet consumption on said user, based on which, treatment plan of said user is dynamically revised.

In yet another aspect, the proposed device can further include a communication module configured to communicate droplet consumption data in real-time or on as required basis to one or more secondary users.

In another aspect, the liquid medicament can be a combination of one or more liquids that are combined in a defined ratio prior to vaporization.

In yet another aspect, size of the generated droplets can be controlled by any or a combination of execution of a set of instructions that are stored in memory unit of the device, or through an application configured in a portable computing device that the drop dispenser device is operatively coupled with.

The present disclosure relates to a method comprising: holding, in a liquid reservoir of a drop dispenser device, a liquid medicament to be vaporized; adapting the liquid medicament to pass through a dispensing opening during a dispensing operation of the drop dispenser device; and generating, based on configuration of a dispensing mechanism that is configured in the drop dispenser device, predetermined droplets of the liquid medicament so as to expel the generated droplets through the dispensing opening in a selected liquid amount and at a selected frequency, upon activation of the dispensing mechanism.

In another aspect, the size of the generated droplets can be controlled by any or a combination of execution of a set of instructions/signals that can be stored in memory unit of the device, or through an application/app configured in a portable computing device (such as a mobile/smart phone, wearable device, laptop, etc), that the drop dispenser device is operatively coupled with, for instance, through Wi-Fi, wireless connection, or Bluetooth, etc.

In another aspect, configuration of the dispensing mechanism can be controlled through a portable computing device that can operatively be coupled with the drop dispenser device through a wireless connection in a manner such that signals for configuration of any or a combination of droplet size, liquid concentration in each droplet, number of droplets to be dispensed, frequency of dispensation, duration of dispensation in each iteration, are transmitted from the portable computing device and processed through a micro-controller configured in the drop dispenser device. The portable computing device can be coupled with a plurality of drop dispenser devices, each drop dispenser device of said plurality drop dispenser devices being associated with a unique identifier that is used in the operational instructions transmitted by said portable computing device to enable identification of the drop dispenser device for which the operational instructions are meant. In an aspect, the operational instructions can be encrypted by said portable computing device, and decrypted using a decryption key by the drop dispenser device.

According to one aspect of the present disclosure, there is provided a drop dispenser capable of dispensing precise, replicable and consistent metered drops of liquid medicament for subsequent vaporization, and ensures that the dispensed drops can be vaporized in a manner that provides patients with a predictable dose. In an aspect, the drop dispenser can include: a liquid reservoir for holding a liquid medicament to be vaporized; a dispensing opening through which the liquid medicament is adapted to pass during a dispensing operation of the dispenser; and a dispensing mechanism configured to generate predetermined droplets of medicament and expel the droplets through the dispensing opening in a selected liquid amount and a selected frequency, upon activation of the dispensing mechanism.

In an embodiment, the drop dispenser disclosed herein can dispense accurately metered drops of herbal oil on a consistent and replicable basis to a heating assembly for subsequent evaporation.

In an aspect, the drop dispenser disclosed herein can incorporate smart features to monitor and record size and number of drops dispensed for vaporization and number of doses remaining in the dispenser thereby enhancing patient clinical care. The maintained records can help in allowing doctors and patients to gain a better understanding of how the medication is functioning to develop a customized treatment program.

In an aspect, the disclosed drop dispenser can further incorporate means to communicate data in real time or on as required basis to various stakeholders such as doctor, care giver, drug stores, pharmaceutical companies etc. The means for communication can be a wireless communication means that can enable data transfer from the drop dispenser to external functional units or networks. Communication between the drop dispenser and external functional units or networks may be accomplished through any suitable communication channels such as Bluetooth, Wi-Fi, GSM communication means such as 3G etc.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure.

FIG. 1 illustrates an exemplary block diagram indicating various functional parts/modules of a smart drop dispenser in accordance with embodiments of the present disclosure.

FIG. 2 illustrates an exemplary representation of the proposed device/dispenser in accordance with an embodiment of the present invention.

FIG. 3 illustrates an exemplary flow diagram showing working of the proposed drop dispenser device in accordance with an invention of the present disclosure.

### DETAILED DESCRIPTION OF THE INVENTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Unless the context requires otherwise, throughout the specification which follow, the word “comprise” and variations thereof, such as, “comprises” and “comprising” are to be construed in an open, inclusive sense that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

The headings and abstract of the invention provided herein are for convenience only and do not interpret the scope or meaning of the embodiments.

Various terms as used herein are shown below. To the extent a term used in a claim is not defined below, it should be given the broadest definition persons in the pertinent art have given that term as reflected in printed publications and issued patents at the time of filing.

According to one aspect of the present disclosure, there is provided a drop dispenser capable of dispensing precise, replicable and consistent metered drops of liquid medication for subsequent vaporization, and ensures that the dispensed drops can be vaporized in a manner that provides patients with a predictable dose. In an aspect, the drop dispenser can include a hollow container body defining a liquid reservoir and provided with a dispensing opening. The liquid reservoir can be configured to hold a volume of liquid medicament that is to be vaporized. In an embodiment, the liquid reservoir can be designed as an exchangeable or replaceable container, so that when the liquid medicament is depleted, it can be replenished in a simple manner. Alternatively, the liquid reservoir can be constructed to be refillable such as by a refill hole. Further, the liquid reservoir can include a dispensing opening for drop-wise release of liquid medicament during a dispensing operation of the dispenser. The dispensing opening can be sized and shaped such that the liquid medicament contained in the liquid reservoir does not drop-release from the dispensing opening unless the drop dispenser is activated.

The disclosed drop dispenser can further incorporate a dispensing mechanism that can be configured for generating predetermined droplets from a liquid medicament stored in the reservoir and expelling the droplets through the dispensing opening in a selected liquid amount and a selected frequency, upon activation of the dispensing mechanism. Thus the dispenser can precisely control size of the droplets to be dispensed thus maintaining a precise control on the dose. Further, the dispensing mechanism can generate droplets of consistent volume and force the metered droplets directly onto a heating assembly for vaporization, and it does not rely on gravity nor on wicking to separate or propel the droplets.

As used herein the term “liquid medicament” or “medicated liquid” encompasses all types of vaporizable liquid substances that have a beneficial or therapeutic effect. Non-limiting examples of liquid medicament suitable for use in the present invention can include any of the oils derived from medicinal herbs and biologically active agents in liquid form.

In an embodiment, the dispensing mechanism can be configured to dispense droplets of liquid medicament in measured amounts, for example droplets of a size of 1 to 100 microliters. The configuration of the drop dispenser ensures that metered droplets exit the dispenser through the dispensing opening thereof regardless of the orientation of the drop dispenser, thereby ensuring operation of the drop dispenser in any orientation. Further, the disclosed drop dispenser can be designed so as to be precisely fit into a dose vaporizer so as to provide for optimal vaporization of liquid medicament and consistent and predictable dose delivery.

In an embodiment, the drop dispenser disclosed herein can dispense accurately metered drops of herbal oil on a consistent and replicable basis to a heating assembly for subsequent evaporation.

In an aspect, the present disclosure relates to a drop dispenser device comprising a liquid reservoir for holding a liquid medicament to be vaporized; a dispensing opening through which the liquid medicament is adapted to pass during a dispensing operation of the dispenser; and a dispensing mechanism configured to generate predetermined droplets of the liquid medicament, and expel the generated droplets through the dispensing opening in a selected liquid amount and at a selected frequency, upon activation of the dispensing mechanism.

In an aspect, drop dispenser can dispense accurate metered droplets of the liquid medicament on a consistent and replicable basis to a heating assembly for subsequent evaporation.

In another aspect, the liquid medicament can be a combination of one or more herbal oils.

In another aspect, the device can be enabled to monitor and record size and number of droplets dispensed for vaporization and number of doses remaining in the dispenser.

In another aspect, the device can be enabled to maintain records of droplet consumption on a central storage device such as on a server/cloud, wherein the records can be accessible from the central storage device to one or more stakeholders based on permissions granted by user of said device. Additionally, in an aspect, the records can be automatically processed to enable determination of impact of droplet consumption on said user, based on which, treatment plan of said user is dynamically revised.

In another aspect, the device can further include a communication module configured to communicate droplet consumption data in real-time or on as required basis to one or more secondary users.

In yet another aspect, the liquid medicament can be a combination of one or more liquids that are combined in a defined ratio prior to vaporization.

In yet another aspect, size of the generated droplets can be controlled by any or a combination of execution of a set of instructions that are stored in memory unit of the device, or through an application configured in a portable computing device that the drop dispenser device is operatively coupled with.

In an aspect, the present disclosure relates to a method comprising the steps of holding a liquid medicament to be vaporized in a liquid reservoir of a drop dispenser device; adapting the liquid medicament to pass through a dispensing opening during a dispensing operation of the drop dispenser device; and generating, based on configuration of a dispensing mechanism that is configured in the drop dispenser device, predetermined droplets of the liquid medicament so as to expel the generated droplets through the dispensing opening in a selected liquid amount and at a selected frequency, upon activation of the dispensing mechanism.

In an aspect, the present disclosure relates to a drop dispenser device comprising: a liquid reservoir for holding a liquid medicament to be vaporized; a dispensing opening through which the liquid medicament is adapted to pass during a dispensing operation of the dispenser; and a dispensing mechanism configured to generate predetermined droplets of the liquid medicament, and expel the generated droplets through the dispensing opening in a selected liquid amount and at a selected frequency, upon activation of the dispensing mechanism.

In another aspect, the drop dispenser dispenses accurate metered droplets of the liquid medicament on a consistent and replicable basis to a heating assembly for subsequent evaporation. In another aspect, the liquid medicament can be a herbal oil such as cannabis oil/extract.

In another aspect, the device can enable monitoring and recording of size and number of droplets dispensed for vaporization and number of doses remaining in the dispenser.

In yet another aspect, the device can enable maintenance of records of droplet consumption on a central storage device such as on a cloud/server, wherein the records are accessible, from the central storage device, to one or more stakeholders based on permissions granted by user of said device.

In another aspect, the records can be automatically processed to enable determination of impact of droplet consumption on said user, based on which, treatment plan of said user is dynamically revised.

In yet another aspect, the proposed device can further include a communication module configured to communicate droplet consumption data in real-time or on as required basis to one or more secondary users.

In another aspect, the liquid medicament can be a combination of one or more liquids that are combined in a defined ratio prior to vaporization.

In yet another aspect, size of the generated droplets can be controlled by any or a combination of execution of a set of instructions that are stored in memory unit of the device, or through an application configured in a portable computing device that the drop dispenser device is operatively coupled with.

The present disclosure relates to a method comprising: holding, in a liquid reservoir of a drop dispenser device, a liquid medicament to be vaporized; adapting the liquid medicament to pass through a dispensing opening during a dispensing operation of the drop dispenser device; and generating, based on configuration of a dispensing mechanism that is configured in the drop dispenser device, predetermined droplets of the liquid medicament so as to expel the generated droplets through the dispensing opening in a selected liquid amount and at a selected frequency, upon activation of the dispensing mechanism.

In another aspect, the size of the generated droplets can be controlled by any or a combination of execution of a set of instructions/signals that can be stored in memory unit of the device, or through an application/app configured in a portable computing device (such as a mobile/smart phone, wearable device, laptop, etc), that the drop dispenser device is operatively coupled with, for instance, through Wi-Fi, wireless connection, or Bluetooth, etc.

In another aspect, configuration of the dispensing mechanism can be controlled through a portable computing device that can operatively be coupled with the drop dispenser device through a wireless connection in a manner such that signals for configuration of any or a combination of droplet size, liquid concentration in each droplet, number of droplets to be dispensed, frequency of dispensation, duration of dispensation in each iteration, are transmitted from the portable computing device and processed through a micro-controller configured in the drop dispenser device. The portable computing device can be coupled with a plurality of drop dispenser devices, each drop dispenser device of said plurality drop dispenser devices being associated with a unique identifier that is used in the operational instructions transmitted by said portable computing device to enable identification of the drop dispenser device for which the operational instructions are meant. In an aspect, the operational instructions can be encrypted by said portable computing device, and decrypted using a decryption key by the drop dispenser device.

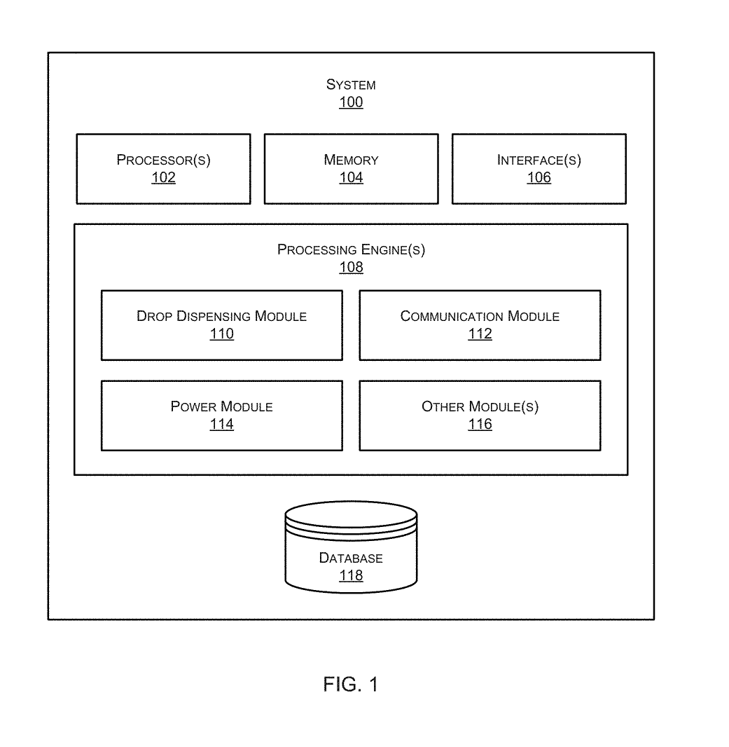

Referring now to FIG. 1, there is shown an exemplary block diagram 100 indicating various functional subsystems of a drop dispenser 100. As illustrated, the proposed device/system 100 can include one or more processor(s) 102 (also interchangeably referred to as micro-controllers). The one or more processor(s) 102 can be implemented as one or more microprocessors, microcomputers, microcontrollers, digital signal processors, central processing units, logic circuitries, and/or any devices that manipulate data based on operational instructions. Among other capabilities, the one or more processor(s) 102 are configured to fetch and execute computer-readable instructions stored in a memory 104 of the system 100. The processors 102 can further be configured to receive instructions from a portable computing device such as a smart phone that may be operatively coupled with the drop dispenser 100, and accordingly follow the dispensing process through vaporization of liquid(s) contained in the dispenser 100, wherein the vaporization, dispensing, drop generation, among other functional aspects of the proposed dispenser 100 can be configured/controller/monitored/analyzed at the portable computing device.

The memory 104 can store one or more computer-readable instructions or routines, which may be fetched and executed to create or share the data units over a network service. The memory 104 can include any non-transitory storage device including, for example, volatile memory such as RAM, or non-volatile memory such as EPROM, flash memory, and the like. In an example embodiment, the memory 104 may be a local memory or may be located remotely, such as a server, a file server, a data server, and the Cloud.

The system 100 can also include an interface(s) 106. The interface(s) 106 may include a variety of interfaces, for example, interfaces for data input and output devices, referred to as I/O devices, storage devices, and the like. The interface(s) 106 may facilitate communication of the system 100 with various devices coupled to the system 100. The interface(s) 106 may also provide a communication pathway for one or more components of the system 100. Examples of such components include, but are not limited to, processing engine(s) 108 and data 118.

The engine(s) 108 can be implemented as a combination of hardware and programming (for example, programmable instructions) to implement one or more functionalities of the engine(s) 108. In examples described herein, such combinations of hardware and programming may be implemented in several different ways. For example, the programming for the engine(s) may be processor executable instructions stored on a non-transitory machine-readable storage medium and the hardware for the engine(s) 108 may include a processing resource (for example, one or more processors), to execute such instructions. In the present examples, the machine-readable storage medium may store instructions that, when executed by the processing resource, implement the engine(s) 108. In such examples, the system 100 can include the machine-readable storage medium storing the instructions and the processing resource to execute the instructions, or the machine-readable storage medium may be separate but accessible to system 100 and the processing resource. In other examples, the engine(s) 108 may be implemented by electronic circuitry. The data 118 can include data that is either stored or generated as a result of functionalities implemented by any of the components of the engine(s) 108.

In an example, the processing engine(s) 108 can include Drop Dispensing Module 110, a Communication Module 112, a Power module 114, and other module(s) 116. The other module(s) 116 can implement functionalities that supplement applications or functions performed by the system 100 or the processing engine(s) 108.

As shown, the smart drop dispenser 100 can include a drop dispensing unit/module 110 that may be constructed in a manner similar to the drop dispenser as described herein in other embodiments. The proposed/smart drop dispenser 100 can further incorporate a communication module 112, a power module 114, and one or more other/additional modules 116.

In an embodiment, the interface module 104 can be configured to permit a user to interact with the drop dispenser 100 to observe size and number of drops dispensed for vaporization, number of doses remaining in the dispenser, account settings, and the like and may also permit the user to control the size of the drops and/or the frequency of drops being dispensed. The interface module 106 (also interchangeably referred to as dispenser interface hereinafter) can also enable user to connect the proposed drop dispenser with external functional units, networks, and the like. In an embodiment, the interface 106 can incorporate a display screen that can be configured to display size and number of drops dispensed from the smart drop dispenser for vaporization. Through the display of the proposed device, an authenticated/verified user can configure liquid medicaments that are to be used for vaporization, proportion of each liquid medicament, size of droplet being generated/formed, number of droplet in each metered dose so that a specific quantity of dose can be consumed, making consumption to be accurate and accounted for. Interface 106 of the proposed device 100 can be a combination of buttons/links/touch-display selectable items or any other means through the user can interact, configure, communicate through the application. In another aspect, the proposed device 100 may not include any interface and may simply be controlled through a mobile application that is configured in a mobile/smart phone, wherein the communication between the proposed dispensing device and the smart phone can be through any or a combination of a Bluetooth module, Wi-Fi, Zigbee protocol, or any other wired/wireless communication means, all of which are well within the scope of the present invention.

In an embodiment, the data storage module 118 (also interchangeably referred to as database 118 hereinafter) can be configured to collect, organize and store information such as size and number of drops dispensed for vaporization and the frequency of drops being dispensed. The stored information can provide records to help doctors and patients to gain a better understanding of how the medication is functioning to develop a customized treatment program. It can also allow pharmaceutical companies and medical researchers to learn about the effectiveness of their medicine and can further allow them to stay connected to the patients using their medicines to gather data and perform analytics. In an aspect, the data storage module/database 118 can be configured in a memory 104 of the propose device, which memory 104 can store consumption pattern of the user and then upload said received data in real-time or at periodic/defined intervals at a central storage device so as to enable remote/central access to one or more stakeholders/secondary users.

In an embodiment, the communication module 112 can be configured to communicate data in real time or on as required basis to various stakeholders such as doctor, caregiver, drug stores, pharmaceutical companies etc. The means for communication can be a wireless communication means that can enable data transfer from the smart drop dispenser 100 to external functional units or networks such as, but not limited to computing devices, mobile devices, networks, databases, other types of application software, etc. Communication between the smart drop dispenser 100 and external functional units or networks may be accomplished through any suitable communication channels such as, but not limited to, Bluetooth, Wi-Fi, GSM communication means such as 3G etc. Thus, the smart drop dispenser 100 and an external functional unit may communicate and provide information and/or instructions one to the other.

In an embodiment, the processor 102 can be functionally coupled to the drop dispensing module 102, the interface 106, the database 118, and the communication module 112. The processor 102 can receive commands from other modules, analyze the received commands, and execute data processing according to the analyzed commands. In an exemplary embodiment, the processor 102 can be a digital signal processor configured to receive and process digital data provided by user in respect of size and number of medicament drops to be dispensed for vaporization, frequency of medicament drops, etc. and based on pre-fed data provide metered drops of medicament for subsequent vaporization.

In an embodiment, the power module 114 can be configured to meet power requirement of various functional subsystems of the smart drop dispenser 100. The power module 114 can include a rechargeable battery with connected charging port and charging circuit.

In an aspect, liquid medicament (simply referred to as liquid hereinafter) can be a combination one of more liquids, each of which can be kept in a separate chamber in the proposed device, and then mixed during the vaporization stage so as to form a droplet that comprises all the liquids in a desired proportion. It is to be appreciated that how much each liquid is to contribute during generation of the droplets can be configured/customized by the user through actuators available on the device itself or remotely through a mobile application that can control working of the proposed device, and also be changed as and when needed so that the right/desired/appropriate concentration of liquid is used and the intended dose of droplet is formed. Such a configuration can either be done by the user himself, or can be done only by the doctor/medical professional who monitors the patients/users treatment progress. Any other configurational change is well within the scope of the present disclosure.

In an aspect, the proposed device can be configured such that it only allows a defined amount of droplets to be generated and consumed by a user/patient based on the treatment regime defined by the doctor, such that upon such consumption, the device can be automatically locked so as to not allow any further generation/dispensation of the droplets.

In another aspect, the proposed device can be operatively coupled with a central computing device such as a server/cloud in a manner such that consumption pattern of a user including quantity consumed, composition of the droplets generated, time of consumption, among other attributes can be recorded in/by the device in real-time and transmitted to the server/cloud. Such a central computing device can then dynamically perform analytics based on the records received from the device and generate one or more reports that can be viewed/analyzed by one or more stakeholders (such as family members, authorized personnel's, and medical practitioners) to assess/evaluate the consumption pattern and take/recommend/suggest rectification steps/feedback to the user or amend/revise the treatment plan.

In yet another aspect, the proposed device can include a touch-interface that can enable the user to control operations parameters of the proposed device such as size of each droplet, quantity being consumed in each iteration of consumption, proportion of combination of liquids that generate the droplets, among other like parameters, all of which are well within the scope of the present disclosure. Such control of the device operation can also be performed using one more actuators presented on the device instead of the touch-based interface, or can be controlled through audio based signals.

In yet another aspect, the proposed device can further include or be operatively coupled with a biometric means that is configured to authenticate the user before the consumption of droplets, said biometric means being based on any or a combination of fingerprint scanner, iris scanner, voice based authentication system, face recognition system, breath analysis based authentication system, among any other biometric technique that can then authenticate the user, post which the liquid medicament is vaporized into droplets for consumption.

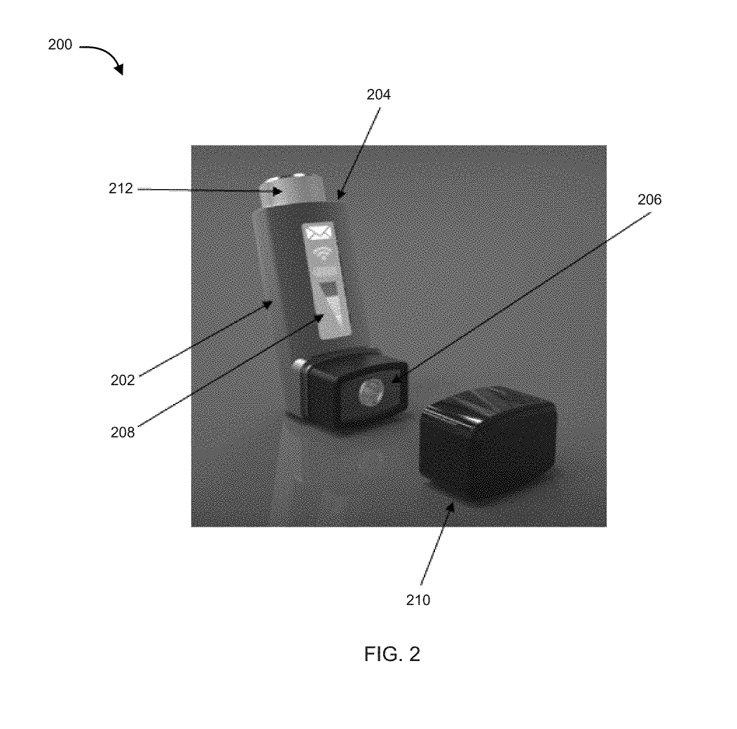

FIG. 2 illustrates an exemplary representation of the proposed device/dispenser 200 in accordance with an embodiment of the present invention. As shown, the proposed device/dispenser 200 can include a housing 202, wherein the housing 202 can include a mouthpiece end 206 at one end, and a receiving end 204 at the other end. The receiving end 204 can be adapted to receive a pressurized aerosol container 212 (interchangeably referred to as container in the present specification/disclosure) containing a predetermined number of unit doses of medication. The aerosol container 212 can include a chamber such that upon actuation of the container by being depressed from the top, metered doses of medication in aerosol are discharged form through the mouthpiece 206 for inhalation by a patient The exemplary screen 208 provided on the housing discharge 202 of the inhaler can indicate status of Wi-Fi, GSM connectivity, effectiveness of medication dose and battery status. The screen 208 can be further used to display and/or enter different data and information that the inhaler may provide or may be required to process or store. The cap 210 can be used to cover the mouthpiece 206 to prevent contaminants from entering the interior of the inhaler and to keep the mouthpiece clean.

In an aspect, the proposed device/dispenser 200 can include a liquid reservoir such as the container 212 for holding a liquid medicament to be vaporized; a dispensing opening configured, for instance, in the mouthpiece 206, through which the liquid medicament is adapted to pass during a dispensing operation of the dispenser 200; and a dispensing mechanism configured to generate predetermined droplets of the liquid medicament, and expel the generated droplets through the dispensing opening in a selected liquid amount and at a selected frequency, upon activation of the dispensing mechanism.

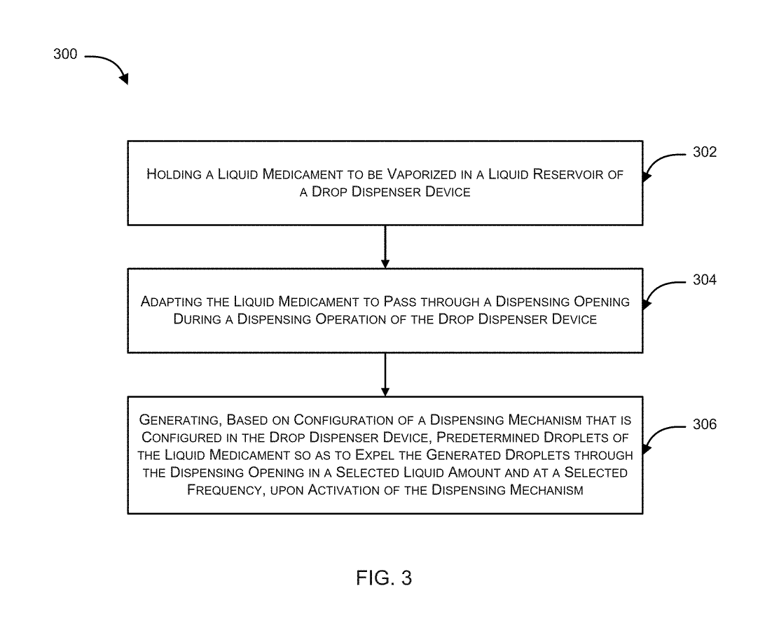

FIG. 3 illustrates an exemplary flow diagram showing working of the proposed drop dispenser device in accordance with an invention of the present disclosure. As shown, step 302 includes holding a liquid medicament to be vaporized in a liquid reservoir of a drop dispenser device; step 304 includes adapting the liquid medicament to pass through a dispensing opening during a dispensing operation of the drop dispenser device; and step 306 includes generating, based on configuration of a dispensing mechanism that is configured in the drop dispenser device, predetermined droplets of the liquid medicament so as to expel the generated droplets through the dispensing opening in a selected liquid amount and at a selected frequency, upon activation of the dispensing mechanism.

As mentioned above, the proposed device can be communicatively coupled with a portable computing device and receive encoded/encrypted signals/instructions from the computing device so as to perform metered dosing based on the instructions, wherein the encrypted signals/instructions are initially decoded/decrypted by the proposed drop dispenser device. Also, memory configured in the proposed drop dispenser device can store consumption patterns of the user and store them on a cloud for further analysis by various authorized personnel's. Also, the proposed device can include a biometric means to authenticate the user before consumption so that only a defined as much number of droplets are released as are required based on the treatment plan allocated/assigned by the user. Any other second user is not allowed to access or retrieve medication from the proposed drop dispenser device, unless authorized through the proposed drop dispenser.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

1. A drop dispenser device comprising:
a liquid reservoir for holding a liquid medicament to be vaporized;
a dispensing opening through which the liquid medicament is adapted to pass during a dispensing operation of the dispenser; and
a dispensing mechanism configured to generate predetermined droplets of the liquid medicament, and expel the generated droplets through the dispensing opening in a selected liquid amount and at a selected frequency, upon activation of the dispensing mechanism.

2. The drop dispenser device of claim 1, wherein the drop dispenser dispenses accurate metered droplets of the liquid medicament on a consistent and replicable basis to a heating assembly for subsequent evaporation.

3. The drop dispenser device of claim 1, wherein the liquid medicament is herbal oil.

4. The drop dispenser device of claim 1, wherein said device enables monitoring and recording of size and number of droplets dispensed for vaporization and number of doses remaining in the dispenser.

5. The drop dispenser device of claim 1, wherein said device enables maintenance of records of droplet consumption on a central storage device, said records being accessible from said central storage device to one or more stakeholders based on permissions granted by user of said device.

6. The drop dispenser device of claim 5, wherein said records are automatically processed to enable determination of impact of droplet consumption on said user, based on which, treatment plan of said user is dynamically revised.

7. The drop dispenser device of claim 1, wherein the device further includes a communication module configured to communicate droplet consumption data in real-time or on as required basis to one or more secondary users.

8. The drop dispenser device of claim 1, wherein the liquid medicament is a combination of one or more liquids that are combined in a defined ratio prior to vaporization.

9. The drop dispenser device of claim 1, wherein size of the generated droplets is controlled by any or a combination of execution of a set of instructions that are stored in memory unit of the device, or through an application configured in a portable computing device that the drop dispenser device is operatively coupled with.

10. A method comprising:
holding, in a liquid reservoir of a drop dispenser device, a liquid medicament to be vaporized;
adapting the liquid medicament to pass through a dispensing opening during a dispensing operation of the drop dispenser device; and
generating, based on configuration of a dispensing mechanism that is configured in the drop dispenser device, predetermined droplets of the liquid medicament so as to expel the generated droplets through the dispensing opening in a selected liquid amount and at a selected frequency, upon activation of the dispensing mechanism.

11. The method of claim 10, wherein the liquid medicament is an extract of cannabis.

12. The method of claim 10, wherein said device enables monitoring and recording of size and number of droplets dispensed for vaporization and number of doses remaining in the dispenser.

13. The method of claim 10, wherein said device enables maintenance of records of droplet consumption on a central storage device, said records being accessible from said central storage device to one or more stakeholders based on permissions granted by user of said device.

14. The method of claim 10, wherein said records are automatically processed to enable determination of impact of droplet consumption on said user, based on which, treatment plan of said user is dynamically revised.

15. The method of claim 10, wherein the device further includes a communication module configured to communicate droplet consumption data in real-time or on as required basis to one or more secondary users.

16. The method of claim 10, wherein the liquid medicament is a combination of one or more liquids that are combined in a defined ratio prior to vaporization.

17. The method of claim 10, wherein size of the generated droplets is controlled by any or a combination of execution of a set of instructions that are stored in memory unit of the device, or through an application configured in a portable computing device that the drop dispenser device is operatively coupled with.

18. The method of claim 10, wherein the configuration of the dispensing mechanism is controlled through a portable computing device that is operatively coupled with the drop dispenser device through a wireless connection in a manner such that signals for configuration of any or a combination of droplet size, liquid concentration in each droplet, number of droplets to be dispensed, frequency of dispensation, duration of dispensation in each iteration, are transmitted from the portable computing device and processed through a micro-controller configured in the drop dispenser device.

19. The method of claim 18, wherein the portable computing device is coupled with a plurality of drop dispenser devices, each drop dispenser device of said plurality drop dispenser devices being associated with a unique identifier that is used in the operational instructions transmitted by said portable computing device to enable identification of the drop dispenser device for which the operational instructions are meant.

20. The method of claim 19, wherein the operational instructions are encrypted by said portable computing device, and decrypted using a decryption key by the drop dispenser device.
